(19) United States
(12) Patent Application Publication
(10) Pub. No.: US 2020/0305856 A1
(43) Pub. Date: Oct. 1, 2020

(54) ILLUMINATED SURGICAL RETRACTOR

(71) Applicant: International Private Bank LLC, Christiansted, VI (US)

(72) Inventor: Gary Bernstein, Newnan, GA (US)

(21) Appl. No.: 16/649,953

(22) PCT Filed: Oct. 12, 2018
(86) PCT No.: PCT/US2018/055612

§ 371 Date: Mar. 23, 2020

Related U.S. Application Data
(60) Provisional application No. 62/572,364, filed on Oct. 13, 2017.

(51) Int. Cl. A61B 17/02; A61B 90/35

(52) U.S. Cl. CPC A61B 17/0218; A61B 2090/306; A61B 90/35

(57) ABSTRACT

An illuminated surgical retractor that has at least two retractor blades such as elongated retractor blades connected to a handle in a manner that allows the blades to fan out from each other is disclosed. The source of illumination is directly connected to the apparatus either removably or permanently. The source of illumination may be attached to the handle or the retractor blades in several alternate embodiments.

### TECHNICAL FIELD

The disclosure relates to the illumination of surgical spaces and tissues and more specifically, to minimally invasive surgeries.

### BACKGROUND OF THE DISCLOSURE

Adequate illumination of a surgical field is important for a surgeon. Illumination for most surgeries is achieved with an overhead mounted light; however, this illumination method presents limitations. The surgeon's head can block the illumination as he or she moves to obtain a better view of the surgical area. The overhead illumination may also fail to provide light of a sufficient strength to illuminate deeper surgical fields located inside body cavities. This problem becomes more pronounced as surgeons shift towards providing minimally invasive surgeries that utilize smaller openings which do not allow as much light to pass through from the surrounding area. Surgeon head lamps address some of these problems, but they also have limitations. The lamps may be tethered to an energy source, limiting the surgeon's mobility in the operating suite. The lamps are also quite heavy, limiting the amount of time a surgeon can wear it before experiencing neck and shoulder fatigue. Furthermore, the source of light in many overhead lamps and head lamps is from fiber optic light sources which provide an inefficient amount of light and lose light energy over the course of the fiber. As the length of the fiber increases, the amount of light available at the end can decrease. In some cases, this can result in inadequate lighting at the surgical site.

Another source of illumination is provided by internal lighting systems which allow for a light source to be inserted into the surgical incision and thereby illuminate the surgical site internally. These lighting systems limit the workable space in the surgical incision and require the surgeon to manually manipulate and position the light and any other surgical tools he or she may be using, which is cumbersome and inefficient. An assistant may also be required to hold and position the light; however, this alternative places more hands at the surgical site, limiting visibility of the area.

Another illumination source stems from surgical retractors with lighting incorporated into their design. While these retractors address some of the limitations mentioned above, the single blade design of the retractor can result in tissue folding around the retractor blade, reducing or obstructing any light from the retractor blade. Additionally, these retractors may be difficult to hold at the optimal angle for illumination of the desired surgical area.

Consequently, there exists in the field a need for an illumination source that can be placed through a surgical incision to directly illuminate the surgical site. The device described below addresses this need.

The description provided in the background section should not be assumed to be prior art merely because it is mentioned in or associated with the background section. The background section may include information that describes one or more aspects of the subject technology.

### SUMMARY

The following is a brief summary, not intended to identify specific elements or to delineate the scope thereof. Its sole purpose is to present some concepts in a simplified form as a prelude to the more detailed description.

One aspect of the subject technology is to provide an apparatus that illuminates a surgical field while retracting tissue away from the surgical site. The apparatus includes two or more retractor blades that are connected to a handle, allowing the retractor blades to rotate, and a light-emitting source attached to the same end of the handle as the retractor blades.

In another aspect, the apparatus has a handle, two or more retractor blades connected to the handle, allowing the retractor blades to rotate, and a light-emitting source attached to each retractor blade. Each retractor blade may have a single light source, or a plurality of light sources distributed along the length of the retractor blade.

In another aspect, the apparatus has a bendable handle or angled handle, two or more retractor blades that are connected to the handle, allowing the retractor blades to rotate, and a light-emitting source. The bendable handle may be manipulated to allow for comfortable holding of the illuminated retractor. Alternatively, the handle may be angled and/or cast in a predetermined optimal angle.

In another aspect, the apparatus has a handle designed to conform to the hand of the user, two or more retractor blades connected to the handle, allowing the retractor blades to rotate, and a light-emitting source.

In yet another aspect, the apparatus has a handle, two or more retractor blades that are connected to the handle in such a manner as to allow the retractor blades to rotate, and a removable light-emitting source. The light-emitting source may be removed for various reasons, such as sterilization.

In an additional aspect, the apparatus has a handle, two or more angled retractor blades connected to the handle allowing the retractor blades to rotate, and a light-emitting source. The retractor blades are angled in a manner that reflects the light off the surface of the retractor blade and creates a dispersed illumination of the surgical field.

In accordance with various aspects of the subject technology, a surgical retractor is provided that includes a handle with a distal end and a proximal end, two or more blades movably attached to the distal end of the handle, and a light source connected to the distal end of the handle.

In accordance with other aspects of the subject technology, a surgical retractor is provided that includes a handle with a distal end and a proximal end, and at least two blades movably attached to the distal end of the handle. Each of the at least two blades have a first surface for abutting a margin of a surgical cavity and a second surface. The surgical retractor also includes a light source removably connected to the second surface of at least one of the blades.

In accordance with other aspects of the subject technology, a surgical retractor is provided that includes a handle with a distal end and a proximal end, and two or more blades, each blade having a first end and a second end. The first ends of the blades are connected to the distal end of the handle in a manner that allows the second ends of the blades to fan out from each other. The surgical retractor also includes at least one light source connected to each blade.

Still other aspects of the subject technology will become readily apparent to those skilled in the art from the following description wherein further embodiments of the subject technology are shown and described. As it will be realized, the subject technology is capable of other different embodiments, and its several details are capable of modifications in various aspects, all without departing from the scope of the subject technology. Accordingly, the drawings and descriptions are illustrative in nature and not restrictive.

### BRIEF DESCRIPTION OF THE DRAWINGS

Various exemplary embodiments of the subject technology will be described in detail, wherein like reference numerals refer to identical or similar components, with reference to the following figures.

FIG. 1 illustrates a top view of an apparatus, in accordance with various aspects of the disclosure.

FIG. 2 illustrates a top view of the apparatus of FIG. 1 with the blades in another configuration, in accordance with various aspects of the disclosure.

FIG. 3 illustrates a top view of a portion of the apparatus of FIG. 1, in accordance with various aspects of the disclosure.

FIG. 4 illustrates a side view of the handle of the apparatus of FIG. 1, in accordance with various aspects of the disclosure.

FIG. 5 illustrates a bottom view of a portion of the apparatus of FIG. 1, in accordance with various aspects of the disclosure.

FIG. 6 illustrates a side view of the apparatus of FIG. 1, in accordance with various aspects of the disclosure.

FIG. 7 illustrates a top view of another implementation of the light-emitting source of the apparatus of FIG. 1, in accordance with various aspects of the disclosure.

FIG. 8 illustrates a top view of another implementation of the handle of the apparatus of FIG. 1, in accordance with various aspects of the disclosure.

FIG. 9 illustrates a side view of another implementation of the blades of the apparatus of FIG. 1, in accordance with various aspects of the disclosure.

FIG. 10 illustrates a top view of another implementation of the blade of the apparatus of FIG. 1, in accordance with various aspects of the disclosure.

FIG. 11 illustrates a bottom view of another implementation of the light-emitting source of the apparatus of FIG. 1, in accordance with various aspects of the disclosure.

FIG. 12 illustrates a bottom view of another implementation of the light-emitting source of the apparatus of FIG. 1, in accordance with various aspects of the disclosure.

FIG. 13 illustrates a side view of another implementation of the light-emitting source of the apparatus of FIG. 1, in accordance with various aspects of the disclosure.

### DETAILED DESCRIPTION

The claimed subject matter is now described with reference to the drawings. In the following description, for purposes of explanation, numerous specific details are set forth in order to provide a thorough understanding of the claimed subject matter. It may be evident, however, that the claimed subject matter may be practiced with or without any combination of these specific details, without departing from the spirit and scope of this subject technology and the claims.

In accordance with various aspects of the disclosure, a surgical retractor is disclosed. The surgical retractor includes a light source that is sometimes referred to herein as a light-emitting source. Similarly, the surgical retractor is sometimes referred to herein as an illuminated surgical retractor. However, it should be appreciated that the light source may be powered on or powered off so that the surgical retractor may or may not currently be emitting light, even if referred to as an illuminated retractor or illuminated surgical retractor with a light-emitting source. The blades may be used for any purpose needed during surgery, and are described herein by way of example as retractor blades.

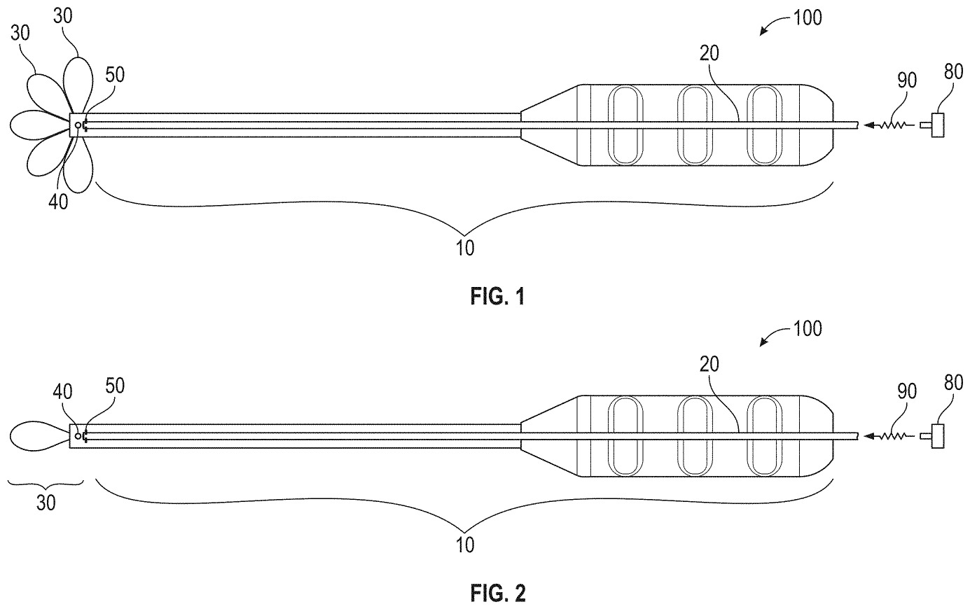

FIG. 1 illustrates an example of a surgical retractor, in accordance with various aspects of the disclosure. As shown in FIG. 1, a surgical retractor such as surgical retractor 100 may be an illuminated retractor that includes a handle 10, two or more retractor blades 30, and a light-emitting source 20 (sometimes referred to herein as a light source). As shown in FIGS. 1 and 2, the retractor blades 30 are attached to the handle 10 in such a manner to allow them to fan out from each other (e.g., to rotate relative to each other about a common pivot axis, which may be defined by a pin 40) between a stacked configuration (FIG. 2) and a fanned-out configuration (FIG. 1). The light-emitting source 20 may be removable and otherwise secured with retainer clips 50.

In the example of FIG. 1, light source 20 comprises an elongate light guide that runs along the length of handle 10. For example, light source 20 may be implemented as a fiber optic light guide that is affixed to handle 10 with retainer clips 50, and that receives and conducts light such as light 90 along handle 10 for emission at or near the location of blades 30 (e.g., for illumination of a surgical site at the location at which the retractor blades are holding back tissue). As indicated in FIG. 1, light 90 may be generated by a light-generating source 80 such as a halogen lamp, one or more light-emitting diodes (LEDs), or other light generators. However, in other implementations, light source 20 may itself be a halogen lamp, LED, or series of LEDs mounted to handle 10 and/or one or more of blades 30 (e.g., using clips 50 or other attachment mechanisms such as adhesive, clamps, or the like).

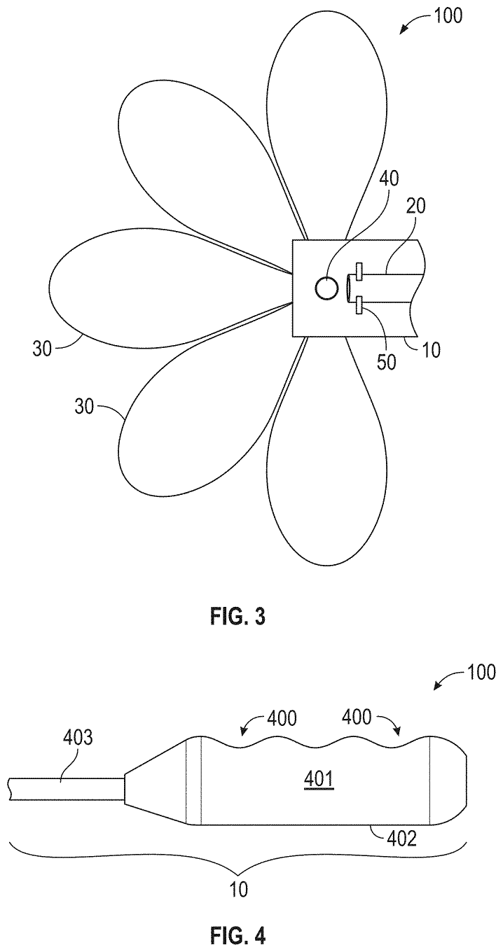

FIG. 3 shows a portion of surgical retractor 100 in the vicinity of pin 40 and retainer clips 50. As shown in FIG. 3, pin 40 may form the rotational (pivot) axis about which blades 30 are rotatable between the stacked configuration shown in FIG. 2 and the fanned-out configuration shown in FIGS. 1 and 3. FIG. 3 also shows how retainer clips 50 may project outward from the top of handle 10 to releasably clip the light-emitting end of light source 20 to handle 10 in the vicinity of blades 30. In the example of FIGS. 1-3, the light-emitting end of light source 20 is set back from the end of handle 10. However, other configurations for the light-emitting end of light source 20 are contemplated and will be described in further detail hereinafter.

FIG. 4 shows a portion of surgical retractor 100 in which features of handle 10 can be seen. The handle 10 can be made of stainless steel, plastic, PVC, alloys or any other bio-compatible material rigid enough to hold back tissue. The handle may be designed to be proportionally wider than an individual retractor blade (e.g., as illustrated and further described hereinafter in connection with FIG. 10), which would allow for more tissue to be held back from the surgical site during retraction and decrease any tenting of tissue around the apparatus, thereby reducing or minimizing the obstruction of emitted light from light source 20.

Handle 10 may be straight, as illustrated in FIGS. 1 and 2, or handle 10 may be bent at a pre-determined angle (e.g., as shown and described in further detail hereinafter in connection with FIG. 8), to enable a user to comfortably hold the apparatus while still illuminating the area of interest. Alternatively, the handle 10 may be constructed in such a manner to allow it to be bendable, so the user may conform the handle to maximize comfort and illumination of the surgical area of interest. For example, in some implementations, a portion of the handle (e.g., the shaft 403 of the handle) may be formed from a pliable material such as a memory plastic, may be formed with at least one pivot point within the handle, or may include any other materials and/or bending mechanisms that would allow the user to change the handle's shape to a desired angle.

Handle 10 may include a grip 401 at the proximal end of the handle (e.g., the proximal end of shaft 403). In the example of FIG. 4, grip 401 is constructed to conform to the hand of the user (e.g., by including indentations 400 that may server as finger holds for a user). Grip 401 may also include a portion 402 (e.g., opposite to indentations 400) that is flat, or cylindrical, or any other shape that provides a gripping surface for a user.

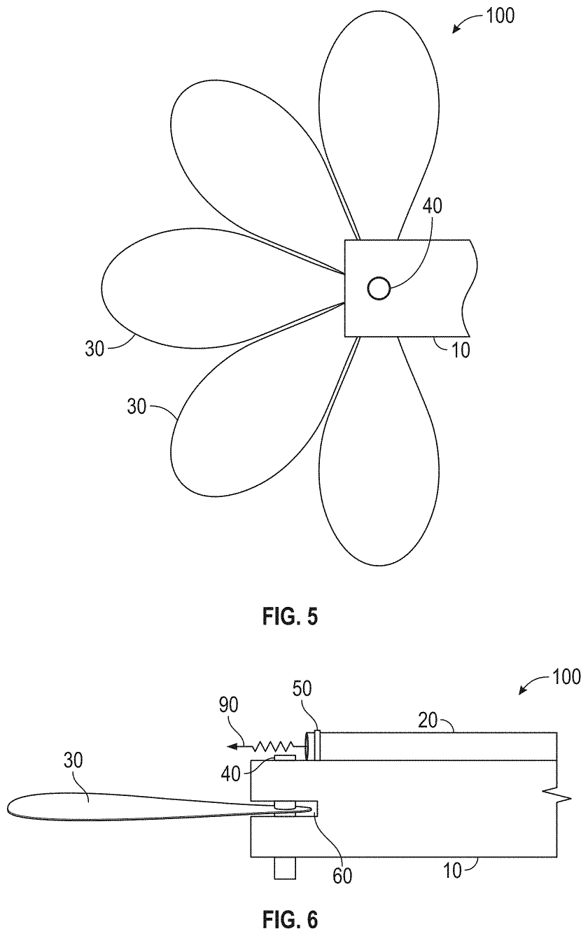

FIGS. 5 and 6 respectively show bottom and side views of surgical retractor 100 according to various aspects of the disclosure. In the bottom view of FIG. 5, a bottom end of pin 40 that provides a rotational axis for blades 30 can be seen.

Retractor blades 30 may be made of stainless steel, plastic, PVC, alloys or any other bio-compatible material rigid enough to hold back tissue. Surgical retractor 100 contains at least two retractor blades 30 but may contain more retractor blades (e.g., five retractor blades 30 as in the examples of FIGS. 1, 3, and 5) without changing the spirit of the subject technology as described herein.

As shown in FIGS. 5 and 6, retractor blades 30 have a distal end and a proximal end. The proximal ends of the retractor blades 30 are connected to the handle 10 in such a manner as to allow the blades to be fanned out from each other, as illustrated in FIG. 1, or compacted, as illustrated in FIG. 2. This allows the retractor blades 30 to fit through a small surgical incision in their compacted form, and then the retractor blades can be expanded inside the surgical cavity to the fanned-out configuration to hold back more tissue from the surgical site.

The retractor blades 30 may be connected to the handle 10 as illustrated in FIG. 6. In the example of FIG. 6, the retractor blades 30 are stacked on top of each other and the proximal ends of the blades are inserted into a notch 60 cut into the distal end of the handle 10. The retractor blades 30 are then held in place with a pin such as pin 40. The pin 40 may be removable, to allow the retractor blades 30 to be removed and either autoclaved or replaced, or the pin may be permanent. Pin 40 may be made of stainless steel, plastic, PVC, alloys or any other bio-compatible material rigid enough to hold the retractor blades 30 in place. The implementation described in connection with FIG. 6 is only an example, and other methods and/or mechanisms for attaching the retractor blades 30 to the handle 10 in a manner that would allow the retractor blades to fan out from each other would be appreciated by those skilled in the art and still within the scope and spirit of the subject technology described herein.

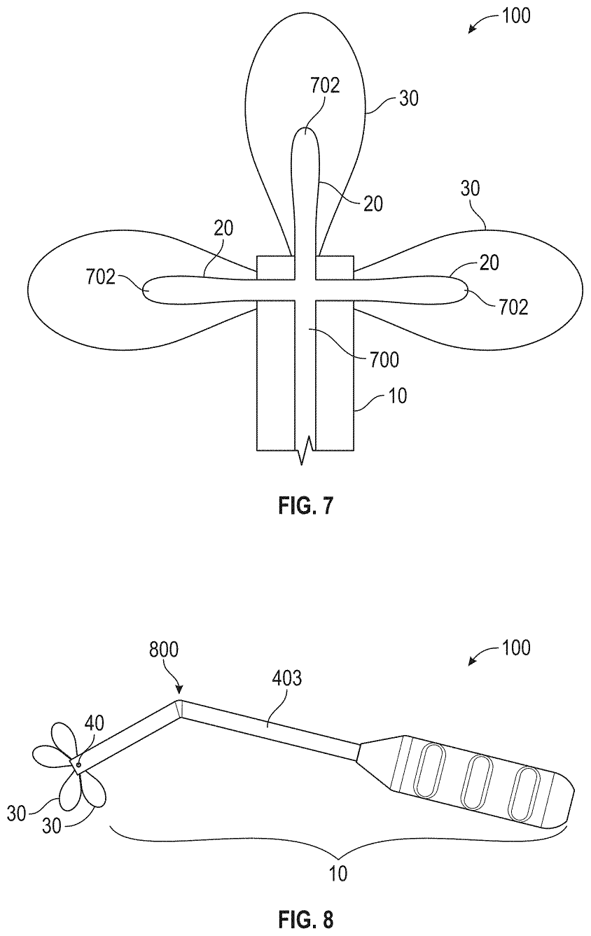

In the example of FIGS. 1-6, a single light source 20 is provided. However, in other implementations, two or more light sources 20 may be provided for surgical retractor 100. For example, FIG. 7 shows an implementation of surgical retractor 100 in which each blade 30 includes a corresponding light source 20. In the example of FIG. 7, surgical retractor 100 includes three blades 30 and three corresponding light sources 20, each mounted to or adjacent to a corresponding blade. However, one, two, four, five, or more than five blades may be provided with one, two, four, five, or more than five corresponding light sources in various implementations.

In the example of FIG. 7, blades 30 and light sources 20 are disposed in a fixed, fanned-out configuration. However, it should be appreciated that blades 30 with dedicated light sources 20 can be rotatable (e.g., with the light sources and blades rotating together) in some implementations. In the example of FIG. 7, all of the blades 30 of surgical retractor 100 have a corresponding (e.g., attached) light source 20. However, in other implementations, one of blades 30, or more than one, but not all, of blades 30 may be provided with corresponding light sources 20.

The light-emitting sources 20 may each be positioned to emit light at the ends of the retractor blades 30, in the middle of the retractor blades (as in the example of FIG. 7), or at any other point along a corresponding retractor blade 30. In another example, the light-emitting portion of one or more light sources 20 may be dispersed along the length of the retractor blade 30, on the side of the retractor blade opposite the side that engages the tissue to be retracted.

In the example of FIG. 7, light sources 20 for each blade 30 are formed from a branch 702 of light guide structure 700 that feeds light along the length of handle 10 to branches 702. However, this is merely illustrative. In other implementations, each light source 20 may be fed from a separate light guide, or may be a stand-alone light source (e.g., an LED) that generates light at the blade.

As described above in connection with, for example, FIG. 4, handle 10 may be straight or bent, in various configurations. FIG. 8 shows an example of surgical retractor 100 with a bent handle. In FIG. 8, light source 20 is not shown for clarity, but may be attached to handle 10 in any of the various configurations described herein. In the example of FIG. 8, shaft 403 of handle 10 includes a bend 800. Bend 800 may be a bend of a fixed or variable angle. For example, bend 800 may be provided when a user bends a shaft 403 that is implemented with a pliable material as described herein, or bends shaft 403 using a bending mechanism such as a hinge that allows shaft 403 to be smoothly bent or bent to one of several preselected angles (e.g., by providing shaft 403 with a hinge having a detent mechanism at the location of bend 800). However, it should also be appreciated that shaft 403 may be a rigid straight shaft or a rigid shaft that is bent at a fixed angle in some implementations.

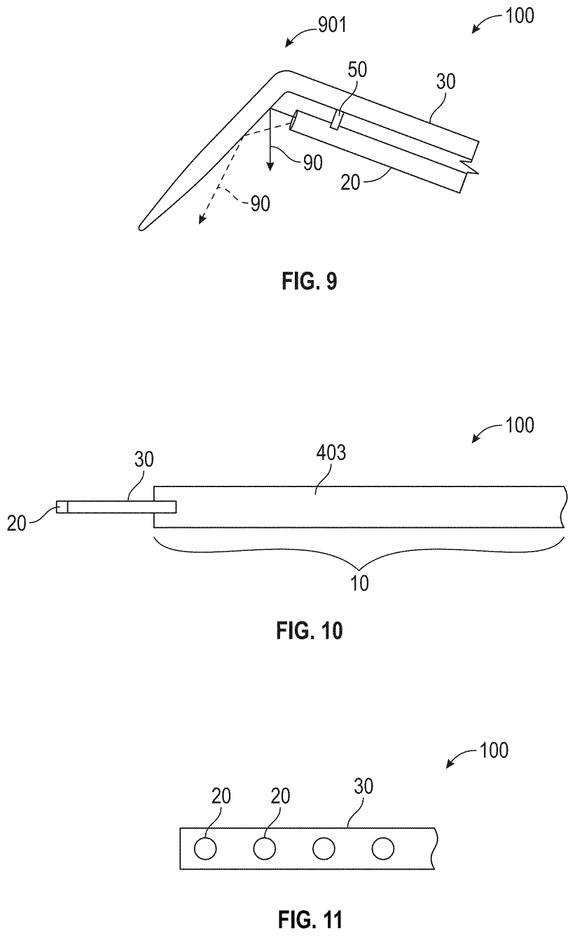

Retractor blades 30 may be flat or planar blades, as illustrated in the example of FIG. 1, or may be bent or curved at a pre-determined angle. FIG. 9 shows an example of a blade 30 having a bend.

As illustrated in FIG. 9, one or more blades 30 may include a bend 901 (e.g., to allow for light 90 coming from the light-emitting source 20 to be reflected by the bent blade onto the surgical site). In accordance with various aspects of the disclosure, each retractor blade 30 may be curved at the same angle, or blades 30 having bends 901 at a variety of angles of pre-determined values may be provided at the distal end of handle 10 to achieve the lighting pattern of interest to the user. The retractor blades 30 may be constructed at the desired angle or the retractor blades may be made of a material (e.g., memory plastic such as a shape-memory polymer) that is pliable enough to allow the user to bend the blades into the desired angle prior to use, and rigid enough after bending to hold back tissue. Bent retractor blades as shown in FIG. 9 may be mounted to handle 10 in a fixed configuration or may be rotatable between stacked and fanned-out configurations as in the examples of FIGS. 1 and 2.

As described above in connection with, for example, FIG. 4, handle 10 may include a shaft 403 that is proportionally wider than an individual retractor blade. FIG. 10 shows a top view of a surgical retractor 100 having a handle 10 that is wider than the blade(s) 30 extending therefrom. In this configuration, more tissue may be held back from the surgical site during retraction, and tenting of tissue around the apparatus may be reduced, thereby reducing or minimizing the obstruction of emitted light from light source 20 (e.g., attached at the end of blade 30 in the example of FIG. 10).

FIG. 11 shows an example of a surgical retractor 100 having multiple light sources 20 for a single blade 30. In the example of FIG. 11, multiple light-emitting sources 20 are dispersed along the length of the retractor blade 30, on the side of the retractor blade opposite the side that engages the tissue to be retracted. In this configuration, the individual light-emitting sources 20 along the length of blade are arranged to illuminate the surgical area collectively. Light sources 20 in the configuration of FIG. 11 can emit light to the surgical area directly and/or by reflection of light off of a part of the retractor blades 30 to which they are attached and/or off of a part of another retractor blade 30 and onto the surgical area (e.g., as described above in connection with FIG. 9). Light emitting sources 20 along the length of blade may be individual, distinct light sources as in the example of FIG. 11, or can be a single elongate light source.

In various examples described above in connection with FIGS. 1-11, one or more light-emitting sources 20 may be attached to the distal portion of the handle 10. In the examples of FIGS. 1-3, 6, 7, and 9-11, light source(s) 20 are mounted on the side of the handle and/or blade that is opposite of the side that engages with the tissue to be retracted from the surgical site. However, in some implementations, one or more light sources 20 may be attached to handle 10 on the same side of the handle as the side that engages with the tissue to be retracted from the surgical site.

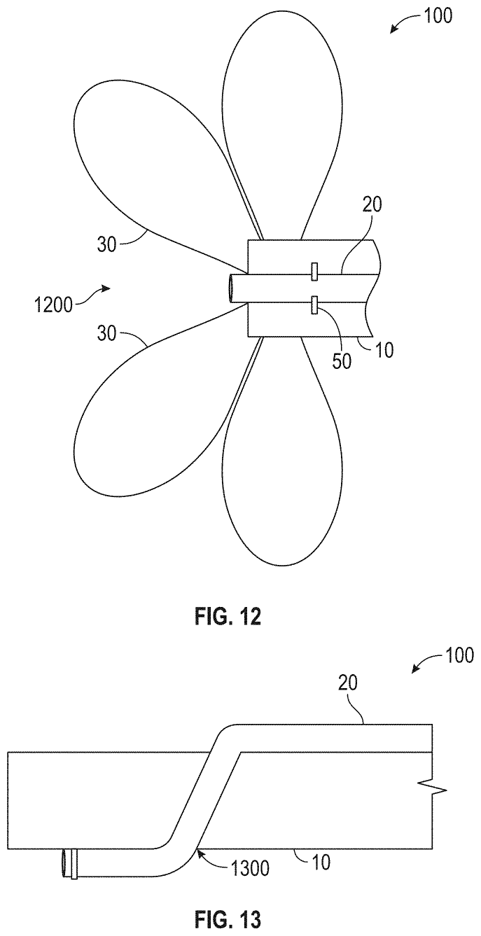

For example, FIG. 12 shows an example in which light source 20 is held to a bottom side of handle 10 using clips 50 mounted to the bottom side. In the example of FIG. 12, light source 20 extends along the same side of the handle 10 as the side that engages with the tissue to be retracted, to a location past the edge (e.g., the distal end) of the handle 10. In this example, the fanned-out configuration of blades 30 may include one or more gaps such as gap 1200 that allows light emitted from light source 20 to pass between two or more blades 30 to illuminate a surgical site.

In another implementation, the light-emitting source 20 may run along the same side of the handle 10 as the side that engages with the tissue to be retracted, and then pass through the handle to the other side, as illustrated in FIG. 13. For example, as shown in FIG. 13, handle 10 may include an opening 1300 that extends from a first side of handle 10 to a second, opposing side of handle 10 that allows light source 20 to pass through handle 10. In the example of FIG. 13, light source 20 is implemented as an elongate light guide (e.g., a fiber optic), and opening 1300 is provided at a soft angle (e.g., non-perpendicular to handle 10) that allows the light guide to curve to pass through handle 10 without breaking. However, in other embodiments, a multi-part light guide may be provided to allow a sharper (e.g., perpendicular) curve in light source 20 than that shown in FIG. 13.

In any of the various configurations described herein the light-emitting source 20 may be a fiber optic, halogen lamp, light-emitting diode, or any other light source which is biocompatible and provides adequate illumination of the surgical site. The light-emitting source 20 may be permanently secured (e.g., to handle 10 and/or one or more blades 30) or the light source may be removable. In one suitable example that is sometimes described herein, the removable light-emitting source 20 is secured to the distal portion of the handle 10 with retainer clips 50. However, it will be appreciated by those skilled in the art that other methods of permanently or removably securing the light-emitting source 20 to the apparatus would be within the scope and spirit of the subject technology.

These embodiments and/or implementations described above are illustrative examples and are not intended to limit the scope of the subject technology described herein as it will be appreciated by those skilled in the art that other variations of the examples described herein would be within the spirit of the subject technology.

A reference to an element in the singular is not intended to mean one and only one unless specifically so stated, but rather one or more. For example, “a” module may refer to one or more modules. An element proceeded by “a,” “an,” “the,” or “said” does not, without further constraints, preclude the existence of additional same elements.

Headings and subheadings, if any, are used for convenience only and do not limit the invention. The word exemplary is used to mean serving as an example or illustration. To the extent that the term include, have, or the like is used, such term is intended to be inclusive in a manner similar to the term comprise as comprise is interpreted when employed as a transitional word in a claim. Relational terms such as first and second and the like may be used to distinguish one entity or action from another without necessarily requiring or implying any actual such relationship or order between such entities or actions.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to the other foregoing phrases.

A phrase “at least one of” preceding a series of items, with the terms “and” or “or” to separate any of the items, modifies the list as a whole, rather than each member of the list. The phrase “at least one” of does not require selection of at least one item; rather, the phrase allows a meaning that includes at least one of any one of the items, and/or at least one of any combination of the items, and/or at least one of each of the items. By way of example, each of the phrases “at least one of A, B, and C” or “at least one of A, B, or C” refers to only A, only B, or only C; any combination of A, B, and C; and/or at least one of each of A, B, and C.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any, present elements of the various steps, operations or processes in a sample order, and are not meant to be limited to the specific order or hierarchy presented. These may be performed in serial, linearly, in parallel or in different order. It should be understood that the described instructions, operations, and systems can generally be integrated together in a single software/hardware product or packaged into multiple software/hardware products.

In one aspect, a term coupled or the like may refer to being directly coupled. In another aspect, a term coupled or the like may refer to being indirectly coupled.

Terms such as top, bottom, front, rear, side, horizontal, vertical, distal, proximal, and the like refer to an arbitrary frame of reference, rather than to the ordinary gravitational frame of reference. Thus, such a term may extend upwardly, downwardly, diagonally, or horizontally in a gravitational frame of reference.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology, and the subject technology is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”.

The title, background, brief description of the drawings, abstract, and drawings are hereby incorporated into the disclosure and are provided as illustrative examples of the disclosure, not as restrictive descriptions. They are submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirements of the applicable patent law, nor should they be interpreted in such a way.

1. A surgical retractor comprising:
a handle with a distal end and a proximal end;
two or more blades movably attached to the distal end of the handle; and
a light source connected to the distal end of the handle, wherein:
the handle is bendable,
the blades are attached to the distal end of the handle with a pin, and
the blades are configured to pivot around the pin.

2. The surgical retractor of claim 1, wherein the light source is a light emitting diode.

3. The surgical retractor of claim 1, further comprising a retainer clip projecting outward from a top of the handle to releasably clip a light-emitting end of the light source.

4. The surgical retractor of claim 1, wherein the proximal end of the handle has a shape that conforms to a shape of a human hand.

5. The surgical retractor of claim 1, wherein the light source is removable from the distal end of the handle.

6. A surgical retractor comprising:
a handle with a distal end and a proximal end;
at least two blades movably attached to the distal end of the handle, wherein each of the at least two blades have a first surface for abutting a margin of a surgical cavity and a second surface; and
a light source removably connected to a second surface of at least one of the blades, wherein:
the light source comprises a fiber optic cable, and
the handle is bendable.

7. The surgical retractor of claim 6, further comprising a retainer clip projecting outward from a top of the handle to releasably clip a light-emitting end of the light source.

8. The surgical retractor of claim 6, wherein the blades are attached to the distal end of the handle in a stack and arranged to be fanned out.

9. The surgical retractor of claim 6, further comprising a pin to secure the blade to the handle and to provide a rotational axis to enable the blade to expand inside a surgical cavity.

10. The surgical retractor of claim 6, wherein the blades are autoclavable.

11. The surgical retractor of claim 6, wherein the at least two blades comprise between two blades and five blades.

12. The surgical retractor of claim 6, wherein the at least two blades are removable from the distal end of the handle.

13. A surgical retractor comprising:
a handle with a distal end and a proximal end;
two or more blades, each blade having a first end and a second end, wherein the first end of the blades are connected to the distal end of the handle in a manner that allows the second end of the blades to fan out from each other; and
at least one light source connected to each blade, wherein:
the handle is bendable,
the blades are attached to the distal end of the handle with a pin, and the blades are configured to pivot around the pin and expand inside a surgical cavity.

14. The surgical retractor of claim 13, wherein the blades are angled to reflect light from the light source onto a surgical field.

15. The surgical retractor of claim 13, wherein the light source comprises a halogen light bulb.

16. The surgical retractor of claim 13, wherein the blades are formed from a material that is reflective to visible light.

17. The surgical retractor of claim 13, wherein the at least one light source that is connected to each blade is removably connected to that blade.

18. The surgical retractor of claim 13, wherein the at least one light source that is connected to each blade is connected to the second end of the blade.

19. The surgical retractor of claim 13, wherein the handle is bent.

20. The surgical retractor of claim 13, wherein the handle is broader than the blades.
